(19) United States
(12) Patent Application Publication
(10) Pub. No.: US 2024/0041718 A1
(43) Pub. Date: Feb. 8, 2024

(54) COMPOSITION COMPRISING AN UNMODIFIED CLAY, A MAGNESIUM SALT, AN ANTISYNERESIS AGENT AND WATER

(71) Applicant: L'OREAL, PARIS (FR)

(72) Inventors: Olga BIGANSKA, CHEVILLY LA RUE (FR); Catherine MARION, CHEVILLY LA RUE (FR); Franck CLEMENT, CHEVILLY LA RUE (FR)

(73) Assignee: L'OREAL, PARIS (FR)

(21) Appl. No.: 18/258,494

(22) PCT Filed: Dec. 21, 2021
(86) PCT No.: PCT/EP2021/087097

§ 371 Date: Jun. 20, 2023

(30) Foreign Application Priority Data
Dec. 22, 2020 (FR) FR2013900

(51) Int. Cl. A61K 8/22; A61K 8/04; A61Q 15/00

(52) U.S. Cl. CPC A61K 8/22; A61K 8/042; A61Q 15/00

(57) ABSTRACT

The present invention relates to a composition, preferably a cosmetic composition, comprising at least: an unmodified clay chosen from trioctahedral smectites, a magnesium salt chosen from magnesium oxide, magnesium hydroxide and their mixtures, an antisyneresis agent chosen from propylene glycol, 1,3-propanediol, natural polymers and their mixtures, and water. It is additionally targeted at a cosmetic method comprising at least one stage which consists in applying such a composition to the skin and/or head hair.

The present invention relates to a composition for topical application comprising at least one unmodified clay chosen from trioctahedral smectites, a magnesium salt, an antisyneresis agent chosen from polyols, natural polymers, organic or nonorganic fillers, crystallizable fatty substances and their mixtures, and water, and to the use of said composition in the field, in particular cosmetic field and especially for the care, the hygiene and/or the protection of keratin materials, such as the skin, in particular of the body or of the face, or head hair, preferably for the care of the skin of the body or of the face.

The present invention relates in particular to the field of cosmetic compositions for the care and hygiene of keratin materials, and especially the skin.

Within the meaning of the present invention, the term “keratin material” is understood to cover the skin, mucus membranes, such as the lips, the nails and keratin fibers, such as the eyelashes and head hair. The skin of the face and/or the skin of the body are very particularly considered according to the invention.

The term “skin” is understood to mean all of the skin of the body, including the scalp, mucus membranes and semimucus membranes, and its appendages. The term “appendages of the skin” is understood to mean body hair, the eyelashes, head hair and the nails. More particularly, in the present invention, the skin of the neckline, of the neck and of the face, and especially the skin of the face, and also the skin of the armpits and of the feet, is considered.

### TECHNICAL FIELD

There is at the current time an increasing consumer demand for cosmetic products or foodstuffs which are termed “natural” because they employ, as essential components, ingredients which are natural, of natural origin and/or certified biological.

Thus, natural products occupy an increasingly important place in the field of skincare and haircare products.

Nevertheless, the performance of natural products is often considered by consumers to be inferior with respect to that of conventional products.

In addition, society today and consumers are also demanding products whose design gives rise to the smallest possible environmental impact.

In this context, the formulator is confronted with a technical challenge as he/she cannot use conventional synthetic starting materials, such as high-performance nonsoaping emulsifiers, thickeners, such as crosslinked or noncrosslinked polyacrylic polymers, or silicones.

Thus, it remains difficult to design natural cosmetic products, the environmental impact of which is as low as possible, which are stable and which have sensory performance qualities equivalent or superior to those of conventional products and specific cosmetic performance qualities, in particular in terms of fresh effect, of deodorant effect (or antiodor effect), of visibility of the pores, of lack of tackiness, lack of stringiness or lack of soaping, or of contribution of mattness.

### PRIOR ART

It is known to use clays in cosmetic products. However, these clays are generally either modified clays, not intended to be used in “natural” products, or natural clays, such as kaolin, talc and mica, but which do not have the property of texturizing water.

There furthermore exist natural clays, such as natural hectorites, which can texturize water by virtue of their specific structure comprising interfoliar cations which can be hydrated, in order to form aqueous gels. These gels are supports that are very advantageous for the design of novel natural cosmetic formulations. Nevertheless, they are not stable over time.

Furthermore, it is also known to use magnesium salts in cosmetic compositions, more particularly in products for greasy skin and in products targeted at combating body odors, however not in the presence of water and of unmodified clays which can texturize it.

### DISCLOSURE OF THE INVENTION

Consequently, users are in search of products which simultaneously meet their expectations in terms of cosmetic quality and sensory nature, which have a reduced environmental impact, and which preferably favor ingredients which are natural, of natural origin and/or certified biological.

In particular, it would be desirable to have available compositions, preferably cosmetic compositions, based on ingredients which are natural, of natural origin and/or certified biological, which are stable and which have sensory performance qualities equivalent or superior to those of conventional products and specific cosmetic performance qualities, in particular in terms of fresh effect, of deodorant effect (or antiodor effect), of lack of tackiness, lack of stringiness or lack of soaping (absence of white film during application), or of contribution of mattness.

It is precisely an object of the present invention to meet these needs.

### SUMMARY OF THE INVENTION

The present text describes a composition, preferably a cosmetic composition, comprising at least:
an unmodified clay chosen from trioctahedral smectites,
a magnesium salt,
an antisyneresis agent chosen from polyols, natural polymers, organic or nonorganic fillers, crystallizable fatty substances and their mixtures, and
water.

According to a first aspect, the present invention is targeted at a composition, preferably a cosmetic composition, comprising at least:
an unmodified clay chosen from trioctahedral smectites,
a magnesium salt chosen from magnesium oxide, magnesium hydroxide and their mixtures,
an antisyneresis agent chosen from propylene glycol, 1,3-propanediol, natural polymers and their mixtures, and
water.

The inventors have discovered, surprisingly, that the compositions according to the invention are stable, and pleasant on application. The joint presence of specific magnesium salt(s) and of specific antisyneresis agent(s) prevents the onset of the well-known phenomenon called syneresis (release of water by the structure of the clay).

In particular, and as illustrated in the examples below, the compositions formed exhibit the advantage of not being tacky or stringy; they are soft, have slip and are fresh on application. Furthermore, they are not soapy; in other words, they do not exhibit a white film during application.

Moreover, they have a mattifying and deodorant performance qualities of a high level.

Finally, their pH is advantageously close to 12, which does not require addition of preservatives.

A further object of the invention, according to another of its aspects, is a cosmetic method comprising at least one stage which consists in applying a composition as defined above to the skin and/or head hair, preferably the skin of the face and/or of the body.

Other characteristics, aspects and advantages of the invention will become apparent on reading the detailed description which will follow.

### DETAILED DESCRIPTION

### Unmodified Clay

As indicated above, a composition according to the invention comprises at least one unmodified clay chosen from trioctahedral smectites.

Generally, the original classification of minerals was that established by the American mineralogist James Dwight Dana (A System of Mineralogy, Durrie & Peck and Herrick & Noyes, 1837, 1^{st} ed.). It was subsequently expanded and a new classification was published in 1997 (Richard V. Gaines, H. Catherine W. Skinner, Eugene E. Foord, Brian Mason and Abraham Rosenzweig, Dana's New Mineralogy, John Wiley & Sons, 1997, 8^{th} ed.).

More particularly, the world reference for the classification of clays is the work by Bergaya/Lagaly (Bergaya F. and Lagaly G., “Handbook of Clay Science”, 2nd Edition. A. Fundamentals—Elsevier Ltd, 2013).

Within the meaning of the present invention, the term “unmodified clay” is understood to mean a natural or synthetic clay which has not undergone any modification of whatever type. For example, when an unmodified clay suitable for the invention is an unmodified hectorite, the latter is distinct from hectorites modified by a C₁₀ to C₂₂ fatty acid ammonium chloride, such as hectorite modified by distearyldimethylammonium chloride.

Thus, an unmodified clay in accordance with the invention is included among the natural or synthetic, preferably natural, phyllosilicates, having a “TOT” (tetrahedron-octahedron-tetrahedron: two tetrahedral sheets framing an octahedral layer) sheet structure, also called 2/1 phyllosilicates, belonging to the group of smectites, and more specifically to the subgroup of trioctahedral smectites.

The structure of the smectites differs from the other phyllosilicates in an interfoliar space between each combination of TOT sheets which depends on its state of hydration (clays sometimes said to be “swelling”) and in which interfoliar cations are intercalated.

In particular, the unmodified clays according to the invention are trioctahedral smectites which can be exfoliated or “activated” in the presence of water, thus forming an aqueous gel as the result of the existence of the interfoliar cations within the structure.

When water comes into contact with an unmodified clay suitable for the invention, it hydrates the sheets of the latter, thus bringing about swelling of the distance between the sheets. A separation of the sheets then takes place, possibly via an exfoliation and/or delamination mechanism. An aqueous gel is then obtained. There is essentially a content ranging from 0.5% to 10% by weight, with respect to the total weight of the composition, of this specific type of clay (combined with the antisyneresis agent as defined in the present invention) which makes it possible to obtain gelled aqueous phases in the compositions according to the invention.

According to one embodiment, an unmodified clay suitable for the invention is synthetic.

According to a preferred embodiment, an unmodified clay suitable for the invention is natural, preferably natural hectorite.

According to a preferred embodiment, an unmodified clay suitable for the invention can comprise at least 30% by weight, preferably from 35% to 65% by weight, of SiO₂, with respect to the total weight of the unmodified clay, and at least 10% by weight, preferably from 15% to 30% by weight, of MgO, with respect to the total weight of the unmodified clay.

According to a more preferred embodiment, an unmodified clay suitable for the invention can comprise from 35% to 65% by weight of SiO₂, with respect to the total weight of the unmodified clay, and from 15% to 30% by weight of MgO, with respect to the total weight of the unmodified clay.

According to a preferred embodiment, a clay suitable for the invention can comprise an SiO₂/MgO ratio by weight ranging from 1 to 3, preferably from 1.5 to 2.5, more preferentially from 1.8 to 2.4.

According to another preferred embodiment, a clay suitable for the invention can comprise from 0% to 15% by weight of Al₂O₃, with respect to the total weight of the unmodified clay.

Thus, according to a first alternative form, a clay suitable for the invention is devoid (denuded) of Al₂O₃.

According to a second alternative form, a clay suitable for the invention can comprise from 0.1% to 15% by weight of Al₂O₃, preferably from 8% to 12% by weight, with respect to the total weight of the unmodified clay.

According to another preferred embodiment, when Al₂O₃ is present within the clay suitable for the invention, the latter can comprise an SiO₂/Al₂O₃ ratio by weight of greater than 3. According to a specific embodiment, an unmodified clay suitable for the invention has as general molecular formula:

$${{[ {M}_{8 - x}^{4 +} {M}_{x}^{3 +} ]}^{tetra} [ {M}_{6 - y}^{2 +} {M}_{y}^{+} ]}^{octa} {{O}_{20} ( OH )}_{4} {X}_{\frac{x + y}{k}}^{k +} \cdot n {H}_{2} O \qquad [ Chem 1 ]$$

in which:
M⁴⁺ represents a cation, preferably Si⁴⁺,
M³⁺ represents a cation, preferably Al³⁺,
M²⁺ represents a cation, preferably Fe²⁺ or Mg²⁺,
M⁺ represents a cation, preferably Li⁺,
X represents an interfoliar cation, preferably Ca²⁺, Na⁺, Li⁺ or their mixtures,
x represents the degree of tetrahedral substitution,
y represents the degree of octahedral substitution,
k represents the valency of the interfoliar cation X, and
n represents an integer, preferably from 0 to 100.

The unmodified clay suitable for the invention is generally available in the powder form. According to a particularly preferred form, the unmodified clay suitable for the invention can be chosen from hectorite, stevensite, saponite and their mixtures, preferably hectorite.

The unmodified clay suitable for the invention can be present in a content of less than or equal to 10% by weight, preferably of less than or equal to 8% by weight, more preferentially of less than or equal to 7% by weight, more preferentially still of less than or equal to 6% by weight, with respect to the total weight of the composition.

In particular, the unmodified clay suitable for the invention is present in a content of between and 5.0% by weight, preferably between 1% and 3% by weight, with respect to the total weight of the composition.

Use can in particular be made, as unmodified clay and more particularly as unmodified hectorite, of that sold by Elementis under the name Bentone EW.

### Water

As set out above, a composition according to the invention additionally comprises water. And, in its presence, an unmodified clay suitable for the invention forms an aqueous gel.

In particular, the water is present in an amount ranging from 30% by weight to 98% by weight, preferably from 35% by weight to 97% by weight and more preferentially from 40% by weight to 95% by weight, with respect to the total weight of the composition.

### Magnesium Salts

As mentioned above, a composition according to the invention additionally comprises at least one magnesium salt which is chosen from magnesium oxide, magnesium hydroxide (Mg(OH)₂), and their mixtures.

According to a particular embodiment, the magnesium salt comprises magnesium oxide, magnesium hydroxide (Mg(OH)₂), or their mixtures.

According to another particular embodiment, the magnesium salt consists in magnesium oxide, magnesium hydroxide (Mg(OH)₂), or their mixtures.

Magnesium salts, and in particular magnesium oxide (MgO), are effective against bad odors. The higher the active concentration, the more effective the product.

Thus, magnesium salts, due to their high basicity, in particular which exhibit a pH of greater than 8 in solution in water, have an antibacterial action and in addition are capable of neutralizing the malodorous compounds of sweat, such as volatile compounds having short carbon chains of the family of the acids.

Use is more particularly made of magnesium oxide.

The magnesium salt, and in particular magnesium oxide, is provided in the form of a more or less dense white powder. As regards the magnesium oxide, it is only very slightly soluble and is more or less rapidly converted into its hydroxide form in the presence of water and can form a gel of a more or less thick texture. It exhibits a highly alkaline pH, in particular of greater than 10.

The magnesium salt, in particular magnesium oxide, powder thus comprises particles which can in particular be characterized by their size, by their density and also by their specific surface.

Mention may in particular be made, among the magnesium oxides which can be employed in the context of the present invention, of the magnesium oxides sold by Ube Industries under the names Ultra High Purity and Fine Magnesium Oxyde 500A, that sold by Dr. Paul Lohmann under the names Magnesium Oxyde Extra Light or Magnesium Oxyde Extra Light Low Nickel, the magnesium oxides sold by Magnesia under the name of Magnesia 22 or also those sold under the name of Magnesium Oxide Heavy C46/124 by Dr. Paul Lohmann.

Mention may in particular be made, as magnesium hydroxide which can be employed in the context of the present invention, of the magnesium hydroxide sold under the name of Magnesium Hydroxide by Dr. Paul Lohmann.

According to a preferred embodiment, a magnesium salt suitable for the invention can be magnesium oxide.

In particular, the magnesium salt(s) suitable for the invention is (are) present in a content of between 0.5% and 10.0% by weight, preferably between 0.9% and 10.0% by weight, with respect to the total weight of the composition.

It is understood that the content of magnesium salt(s) which would possibly be present in an additive as described in detail in the present invention does not come within the percentages of content which are indicated above.

Preferably, when a composition according to the invention is devoted to a product for greasy skin, the magnesium salt(s) suitable for the invention is (are) present in a content of between 0.9% and 5.0% by weight, preferably between 0.9% and 1.5% by weight, with respect to the total weight of the composition.

Preferably, when a composition according to the invention is devoted to a deodorant product, the magnesium salt(s) suitable for the invention is (are) present in a content of between 2% and 10% by weight, preferably between 2% and 8% by weight, with respect to the total weight of the composition.

According to a preferred embodiment, in a composition according to the invention, the unmodified clay/magnesium salt(s) ratio by weight is between 10/1 and 1/10.

In particular, the unmodified clay/magnesium salt(s) ratio by weight is between 10/1 and ½; preferably, it is between 5/2 and ¼. More particularly, it can be 5/2, 2/2, ⅛, ¼ or 1/1.

The use of at least one magnesium salt in a composition according to the invention is advantageous in more than one respect. This is because, insofar as, on the one hand, this/these salt(s) exhibit(s) an intrinsic antibacterial activity and as, on the other hand, its pH is high, it is not necessary to add preservatives in order to provide the compositions according to the invention with microbiological protection.

The thickness and the appearance of the gels formed according to the invention vary both as a function of the content of unmodified clay and of the content of the magnesium salt(s). For example, the greater the content of unmodified clay within a composition according to the invention, the thicker and stiffer the gel obtained.

The addition of a magnesium salt, such as MgO, even in a very small amount, significantly increases the thickness of the gels.

In the case of the combination of small amounts of unmodified clay (for example from 0.5% to 2% by weight, with respect to the total weight of the composition) and of large amounts of magnesium salts, such as MgO (for example from 4% to 5% by weight, with respect to the total weight of the composition), the gels are less thick, more manageable, more creamy than in the case where the content of unmodified clay is higher (for example from 3% to 5% by weight, with respect to the total weight of the composition) and the content of magnesium salts, such as MgO, is lower (for example from 1% to 2% by weight, with respect to the total weight of the composition).

Advantageously, this thus allows the formulator to design prototypes with varied sensory profiles suited to the nature of the future product (cream for the face or deodorant) and to the mode of application (roll-on, balm or stick).

### Antisyneresis Agents

As mentioned above, a composition according to the invention additionally comprises at least one antisyneresis agent chosen from propylene glycol, 1,3-propanediol, natural polymers and their mixtures.

The presence of such agent(s) contributes to the stability of the composition insofar as this prevents the onset of syneresis.

The syneresis is a phenomenon well-known by the skilled in the art which consists of the contraction of the clay gel ejecting a liquid phase from the system.

According to a preferred embodiment, the at least one antisyneresis agent is chosen from propylene glycol, 1,3-propanediol, and their mixtures, preferably propylene glycol. According to a preferred embodiment, in a composition according to the invention, the unmodified clay/antisyneresis agents(s) ratio by weight is between 10/1 and 1/10.

The propylene glycol, 1,3-propanediol, or their mixtures advantageously represent(s) from 1% to 60% by weight, preferably from 2% to 55% by weight, more preferentially from 3% to 50% by weight and better still from 5% to 20% by weight, with respect to the total weight of the composition.

### Natural Polymers

A composition according to the invention can additionally or alternatively to propylene glycol, 1,3-propanediol, or their mixtures comprise at least one natural polymer.

Within the meaning of the present invention, the term “natural polymer” should be understood as meaning any polymer of natural origin, in particular of vegetable or animal origin or resulting from fermentation, preferably chosen from:
modified or native polysaccharides, such as alginates, pectins, carrageenans, starches, in particular modified starches, gellan gum, sclerotium gum, acacia gum or gum arabic resulting from the tree Acacia senegal, locust bean gum, xanthan gum, galactomannans, glucomannans, pullulans, natural or modified scleroglucans, modified or unmodified guar gum, modified or unmodified cellulose, in particular cellulose modified by esterification, etherification (for example CMC, also called carboxymethylcellulose or cellulose gum) and/or crosslinking, hemicellulose, konjac gum, tara gum, karaya gum, hyaluronic acid and its derivatives and/or salts, polyhydroxyalkanoates (PHAs), agar or inulin,
polyamino acids, such as polylysine, and
their mixtures.

According to a preferred embodiment, a natural polymer suitable for the invention can be chosen from carrageenans, alginates, xanthan gum and their mixtures.

According to a specific embodiment, a natural polymer suitable for the invention can be chosen from sclerotium gum, acacia gum, cellulose gum and their mixtures.

The natural polymer(s) suitable for the invention can be present in an amount ranging from 0.05% to 10.00% by weight, preferably from 0.1% to 3.0% by weight, preferably from 0.2% to 2.0% by weight, with respect to the total weight of the composition.

According to a preferred embodiment, in a composition according to the invention, the unmodified clay/natural polymer(s) ratio by weight is of between 0.005/10 and 1/10, preferably between 0.05/10 and 0.3/10.

### Additional Compounds

### Polyols

A composition according to the invention can additionally comprise at least one polyol distinct from propylene glycol and 1,3-propanediol.

Within the meaning of the present invention, the term “polyol” should be understood as meaning any organic molecule comprising at least two free hydroxyl groups.

Preferably, a polyol in accordance with the present invention is present in liquid form at ambient temperature.

A polyol suitable for the invention can be a compound of saturated or unsaturated and linear, branched or cyclic alkyl type carrying, on the alkyl chain, at least two —OH functional groups, in particular at least three —OH functional groups and more particularly at least four —OH functional groups.

The polyols which are advantageously suitable for the formulation of a composition according to the present invention are those exhibiting in particular from 2 to 32 carbon atoms, preferably from 3 to 16 carbon atoms.

Advantageously, the polyol can, for example, be chosen from ethylene glycol, pentaerythritol, trimethylolpropane, butylene glycol, isoprene glycol, pentylene glycol, hexylene glycol, glycerol, polyglycerols, such as glycerol oligomers, for example diglycerol oligomers, polyethylene glycols and their mixtures.

According to a preferred embodiment of the invention, said polyol is chosen from butylene glycol, pentylene glycol, glycerol, polyglycerols, polyethylene glycols and their mixtures.

According to a more preferred embodiment, said polyol is chosen from butylene glycol, glycerol, pentylene glycol and their mixtures.

According to a specific mode, the composition of the invention can comprise at least propylene glycol.

According to another specific mode, the composition of the invention can comprise at least butylene glycol.

The polyol(s) including propylene glycol, 1,3-propanediol, or their mixtures if present, advantageously represent(s) from 1% to 60% by weight, preferably from 2% to 55% by weight, more preferentially from 3% to 50% by weight and better still from 5% to 20% by weight, with respect to the total weight of the composition.

### Fillers

According to one embodiment, the composition according to the invention can additionally comprise at least one filler distinct from an unmodified clay suitable for the invention.

The filler(s) can in particular be organic fillers and/or inorganic (mineral) fillers.

According to one alternative form, the filler is an inorganic filler.

According to another alternative form, the filler is an organic filler.

According to another alternative form, the fillers present in the composition are a mixture of at least one inorganic (mineral) filler and of at least one organic filler.

The term “filler” should be understood as meaning colorless or white and mineral or synthetic particles of any shape which are insoluble in the medium of the composition, whatever the temperature at which the composition is manufactured.

According to a specific embodiment, the fillers used in the present invention exhibit a size, expressed as volume-average diameter D[4,3], ranging from 50 nm to 500 μm. According to a specific embodiment, the fillers used in the present invention exhibit a bulk density of greater than or equal to 0.05 g/cm³.

### Organic Fillers

In the present patent application, the term “organic filler” is understood to mean any organic solid which is insoluble in the medium at ambient temperature (25° C.).

The term “organic” is understood to mean any compound or polymer, the chemical structure of which comprises at least one or more carbon atoms.

Mention may be made, as organic fillers capable of being used in the composition according to the invention, for example, of polyamide (Nylon®) particles and in particular those sold under the names Orgasol® by Atochem; polyethylene powders; microspheres based on acrylic copolymers, such as those made of ethylene glycol dimethacrylate/lauryl methacrylate copolymer sold by Dow Corning under the name of Polytrap®; polymethyl methacrylate microspheres, sold under the name Microsphere M-100® by Matsumoto or under the name Covabead LH85® by Wackherr; ethylene/acrylate copolymer powders, such as those sold under the name Flobeads® by Sumitomo Seika Chemicals; expanded powders, such as hollow microspheres and in particular microspheres formed of a terpolymer of vinylidene chloride, acrylonitrile and methacrylate and sold under the name Expancel® by Kemanord Plast under the references 551 DE 12® (particle size of approximately 12 μm), 551 DE 20® (particle size of approximately 30 μm) or 551 DE 50®) (particle size of approximately 40 μm), or microspheres sold under the name Micropearl F 80 ED® by Matsumoto;
powders of natural organic materials, such as polysaccharide powders and in particular starch powders, especially crosslinked or noncrosslinked corn, wheat or rice starch powders, powders of starch crosslinked by octenylsuccinic anhydride sold under the name Dry-Flo® by National Starch or powders of waxy corn starch, such as those which are sold under the names C* Gel 04201 by Cargill, Maize Starch B by Roquette and Organic Corn Starch by Draco Natural Products; cellulose particles, such as those which are sold under the name Cellulobeads by Daito Kasei Kogyo—mention may also be made of the range of the Tencel products from Lenzing; silicone resin microbeads, such as those sold under the name Tospearl by Toshiba Silicone, in particular Tospearl 240; amino acid powders, such as the lauroyl lysine powder sold under the name Amihope LL-11® by Ajinomoto; wax microdispersion particles which preferably have mean dimensions of less than 25 μm and in particular ranging from 0.5 μm to 25 μm and which are constituted essentially of a wax or of a mixture of waxes, such as the products sold under the name Aquacer by Byk Cera, and in particular: Aquacer 520 (mixture of synthetic and natural waxes), Aquacer 514® or 513® (polyethylene wax) or Aquacer 511® (polymer wax), or such as the products sold under the name Jonwax 120 by Johnson Polymer (mixture of polyethylene and paraffin waxes) and under the name Ceraflour 961® by Byk Cera (micronized modified polyethylene wax); and their mixtures.

According to a specific embodiment of the invention, the organic filler(s) is (are) chosen from spherical cellulose particles, N-acylamino acid powders and polyamide particles; preferably, they are chosen from spherical cellulose particles and N-acylamino acid powders. In the context of the present invention, the term “spherical particles” is understood to mean particles having the shape or substantially the shape of a sphere and which are insoluble in the medium of the composition according to the invention, even at the melting point of the medium (approximately 100° C.). According to a specific embodiment, the spherical cellulose particles which can be used in the context of the invention are microparticles. Preferably, they have a particle size, expressed as volume-average diameter D[4,3], ranging from 0.1 to 35 μm, preferably from 1 to 20 μm and more particularly from 4 to 15 μm.

Mention may in particular be made, as examples of spherical cellulose microparticles, of the solid cellulose beads sold under the names Cellulobeads D-10, Cellulobeads D-5 and Cellulobeads USF by Daito Kasei Kogyo.

The N-acylamino acids can comprise an acyl group having from 8 to 22 carbon atoms, such as, for example, a 2-ethylhexanoyl, caproyl, lauroyl, myristoyl, palmitoyl, stearoyl or cocoyl group. The amino acid can, for example, be lysine, glutamic acid or alanine, preferably lysine.

According to a specific embodiment, the N-acylamino acid(s) comprise(s) an acyl group having from 10 to 14 carbon atoms. Preferably, it is a lauroyl group. Advantageously, the N-acylamino acid powder can be a lauroyl lysine powder, such as that which is sold under the name Amihope LL by Ajinomoto or also that which is sold under the name Corum 5105 S by Corum.

### Inorganic Fillers

In the present invention, the term “inorganic filler” is understood to mean any inorganic solid which is insoluble in the medium at ambient temperature (25° C.).

The term “inorganic” is understood to mean any compound or polymer, the chemical structure of which does not comprise a carbon atom.

Mention may be made, as example of inorganic filler, of porous spherical silica particles having a particle size, expressed as volume-average diameter D[4,3], ranging from 50 nm to 500 μm.

In the present patent application, the term “spherical particles” is understood to mean particles having the shape or substantially the shape of a sphere and which are insoluble in the medium of the composition according to the invention, even at the melting point of the medium (approximately 100° C.).

According to a specific embodiment, they have a specific surface ranging from 30 to 1000 m²/g and more particularly from 150 to 800 m²/g.

According to another specific embodiment, they have an oil absorption capacity ranging from 0.15 to 5 ml/g and more particularly from 1.30 to 1.90 ml/g.

Use may be made, as examples of porous silica microbeads, of the following commercial products: Silica Beads SB-150, SB-300 or SB-700, preferentially SB-300, from Miyoshi Kasei; the range of the Sunsphere products from Asahi Glass AGC Si-Tech, in particular Sunsphere H-51 or Sunsphere 12L, Sunsphere H-201, H-52 and H-53; Sunsil 130 from Sunjin; Spherica P-1500 from Ikeda Corporation;
Sylosphere from Fuji Silysia; the Silica Pearl and Satinier ranges from JGC Catalysts and Chemicals, more particularly Satinier M13 and M16; the MSS-500 silicas from Kobo, and more particularly MSS-500-20N; and also the Silica Shells products from Kobo.

Mention may also be made of zeolites, such as the products sold by Zeochem under the names Zeoflair 300, Zeoflair 200, Zeoflair 100, X-Mol and X-Mol MT,

A zeolite is a crystal formed of a microporous aluminosilicate backbone, the related empty spaces of which are initially occupied by cations and water molecules. They are also described as molecular sieves.

Mention may also be made of calcium magnesium carbonate, such as those which are sold by Imerys under the name Calcidol, by LCW (Sensient) under the name Carbomat or by Omya under the name Omyacare S 60-AV.

Mention may also be made of lamellar inorganic particles, such as talcs, micas and their mixtures.

Talcs are hydrated magnesium silicates generally comprising aluminum silicate. The crystal structure of talc consists of repeated layers of a sandwich of brucite between layers of silica.

More particularly, the lamellar particles are chosen from talcs.

Advantageously, use is more particularly made, in the composition of the invention, as lamellar particles, of talc, such as those sold under the names Luzenac Pharma M and UM by Imrys or Rose Talc and Talc SG-2000 by Nippon Talc.

Mention may be made, among inorganic fillers, of perlite particles and preferably expanded perlite particles.

The perlites which can be used according to the invention are generally aluminosilicates of volcanic origin and have the composition:
by weight of silica SiO₂;
12.0-15.0% by weight of aluminum oxide Al₂O₃;
3.0-5.0% of sodium oxide Na₂O;
3.0-5.0% of potassium oxide K₂O;
of iron oxide Fe₂O₃;
of magnesium oxide MgO;
of calcium oxide CaO;
0.05-0.15% of titanium oxide TiO₂.

Mention may in particular be made of the perlites sold under the names Optimat 2550 OR by World Minerals, and Europerl EMP-2 and Europerl 1 by Imerys.

Thus, according to a specific embodiment, an organic or nonorganic filler suitable for the invention can be chosen from polyamide particles; polyethylene powders; microspheres based on acrylic copolymers; polymethyl methacrylate microspheres; ethylene/acrylate copolymer powders; expanded powders, such as hollow microspheres and in particular microspheres formed of a terpolymer of vinylidene chloride, acrylonitrile and methacrylate; powders of natural organic materials, such as polysaccharide powders and in particular starch powders, especially crosslinked or noncrosslinked corn, wheat or rice starch powders, powders of starch crosslinked by octenylsuccinic anhydride; cellulose particles; silicone resin microbeads; amino acid powders, such as lauroyl lysine powder; wax microdispersion particles; porous silica microbeads; zeolites; calcium magnesium carbonate; perlites; and their mixtures.

These fillers can be present in amounts ranging from 0% to 20% by weight and preferably from 1% to 10% by weight, with respect to the total weight of the composition.

When present, the content of these fillers can range from 0.001% to 20.000% by weight, preferably from 1% to 10% by weight, with respect to the total weight of the composition.

Preferably, the concentrations by weight of filler range from 0.1% to 30.0% and preferably from 0.5% to 15.0% by weight, with respect to the total weight of said composition.

### Crystallizable Fatty Substances

The composition according to the invention can comprise at least one crystallizable fatty substance.

Within the meaning of the present invention, the term “crystallizable fatty substance” is understood to mean a solid lipophilic compound which is or is not deformable at ambient temperature (25° C.) and which exhibits a melting point of greater than or equal to 25° C., preferably of between 25° C. and 200° C., preferably of between 25° C. and 120° C.

Some crystallizable fatty substances are commonly called waxes.

Within the meaning of the invention, the melting point corresponds to the temperature of the most endothermic peak observed on thermal analysis (DSC) as described in the standard ISO 11357-3; 1999. The melting point of the crystallizable fatty substance can be measured using a differential scanning calorimeter (DSC), for example the calorimeter sold under the name MDSC 2920 by TA Instruments. Such a measurement method is, for example, described in the document PCT/EP2013/062964.

Mention may be made, among crystallizable fatty substances of mineral origin, of: paraffin wax, ozokerite, ceresin and microcrystalline wax.

Mention may be made, among crystallizable fatty substances of vegetable origin, of: carnauba wax, candelilla wax, such as that sold under the reference SP 75 G by Strahl & Pitsch, laurel wax, sugar cane wax, ceramide, esparto wax, olive tree wax, rice wax, such as that sold under the reference NC 1720 by Cera Rica Noda, sunflower seed wax, such as that sold by Koster Keunen under the reference sunflower wax, hydrogenated jojoba wax, hydrogenated castor oil, hydrogenated olive oil, hydrogenated cottonseed oil, polyglyceryl-3 esters of Acacia decurrens flower, jojoba and sunflower waxes, and absolute flower waxes, such as blackcurrant blossom essential wax, soybean wax, Myrica fruit wax or laurel wax.

Mention may be made, among crystallizable fatty substances of animal origin, of: beeswaxes or modified beeswaxes (cera bellina), lanolin and spermaceti.

The crystallizable fatty substance(s) can also be chosen from long-chain crystallizable fatty alcohols and their mixtures, such as, for example, cetearyl (C₁₆/C₁₈ 50/50) alcohol, such as that sold under the name Lanette 0 OR by BASF, stearyl alcohol, myristyl alcohol, cetyl alcohol or C₂₆-C₂₂ alcohols.

The crystallizable fatty substance(s) can also be chosen from long-chain crystallizable esters and their mixtures, such as the INCI compound “Cetyl Esters (and) Cetyl Esters Mixture of Myristyl Stearate and Myristyl Palmitate”, or the INCI compound “Mixture of Myristyl Stearate and Myristyl Palmitate”, such as that sold under the name of SP Crodamol MS MBAL-PA-(SG) by Croda, glycol distearate, glycol stearate, cetyl palmitate, such as the commercial product Ercawax CP V/O from the supplier ERCA, isopropyl palmitate, C₂₀-C₄₀ alkyl stearates, long-chain crystallizable esters of glycerol and their mixtures, such as, for example, the compound sold under the name Compritol®888 CG ATO by Gattefossé (INCI: Glyceryl Dibehenate (and) Tribehenin (and) Glyceryl Behenate) or each of its components taken separately, tricaprin, trilaurin, trimyristin, tripalmitin, tristearin, glycerol distearate, glyceryl distearate, glyceryl dipalmitate/stearate and linoleoyl polyoxyl-6 glyceride.

The crystallizable fatty substance(s) can also be chosen from crystallizable fatty acids having a long alkyl chain and their mixtures, such as, for example, the INCI compound “1300 Stearic Acid” (stearic acid), such as in particular that sold under the name of Stearin TP 1200 Pellets (DUB 50 P) from Stéarinerie Dubois, mixtures of stearic acid and palmitic acid, in particular from saturated C₄-C₂₈ fatty acids and unsaturated C₄-C₂₈ fatty acids.

The other crystallizable fatty substances which can be used according to the invention are in particular marine waxes, polyethylene waxes or polyolefin waxes in general, such as α-olefin oligomers, for example the polymers Performa V® 825, 103 and 260 sold by New Phase Technologies, ethylene/propylene copolymers, such as Performalene® EP 700, or Fischer-Tropsch waxes or a mixture of these products.

Thus, according to a specific embodiment, a crystallizable fatty substance suitable for the invention can be chosen from paraffin wax, ozokerite, ceresin, microcrystalline wax, carnauba wax, candelilla wax, laurel wax, sugar cane wax, ceramide, esparto wax, olive tree wax, rice wax, sunflower seed wax, hydrogenated jojoba wax, hydrogenated castor oil, hydrogenated olive oil, hydrogenated cottonseed oil, polyglyceryl-3 esters of Acacia decurrens flower, jojoba and sunflower waxes, absolute flower waxes, such as blackcurrant blossom essential wax, soybean wax, Myrica fruit wax or laurel wax, beeswaxes, modified beeswaxes (cera bellina), lanolin, spermaceti, long-chain crystallizable fatty alcohols and their mixtures, long-chain crystallizable esters and their mixtures, crystallizable fatty acids having a long alkyl chain and their mixtures, marine waxes, polyolefin waxes, ethylene/propylene copolymers, Fischer-Tropsch waxes and their mixtures.

Advantageously, the crystallizable fatty substance is chosen from crystallizable C₁₂-C₂₄ fatty alcohols, crystallizable C₁₂-C₂₄ fatty acids, crystallizable esters resulting from C₁₂-C₂₄ fatty acids and their mixtures.

According to a preferred embodiment, the crystallizable fatty substance is chosen from cetearyl (C₁₆/C₁₈ 50/50) alcohol, mixtures of cetyl esters, mixture of Myristyl Stearate and Myristyl Palmitate, stearic acid and their mixtures.

Preferably, the crystallizable fatty substance is chosen from crystallizable esters resulting from C₁₂-C₂₄ fatty alcohols and/or crystallizable esters resulting from C₁₂-C₂₄ fatty acids, and their mixtures, preferably chosen from cetyl palmitate and a mixture of esters obtained from glycerol and behenic acid.

Preferably, the crystallizable fatty substance is present in a content of between 0.01% and 15.00% by weight, with respect to the weight of the composition, preferably between and 12.00% by weight, preferentially between 0.1% and 8.0% by weight, advantageously between 0.1% and 5.0% by weight.

According to a specific embodiment, when the crystallizable fatty substance is a long-chain crystallizable fatty alcohol or a mixture of such fatty alcohols, it can be comprised in a content ranging from 0.1% and 10.0% by weight, with respect to the total weight of the composition, preferably between 0.5% and 5.0% by weight, preferentially between 1% and 4% by weight, better still 3% by weight.

According to another specific embodiment, when the crystallizable fatty substance is a crystallizable fatty acid having a long alkyl chain or a mixture of such fatty acids, it can be comprised in a content ranging from 0.01% and 5.00% by weight, with respect to the total weight of the composition, preferably between 0.1% and 1.0% by weight, preferentially between 0.1% and 0.3% by weight, better still 0.2% by weight.

According to another specific embodiment, when the crystallizable fatty substance is a long-chain crystallizable ester or a mixture of such esters, it can be comprised in a content ranging from 0.1% and 5.0% by weight, with respect to the total weight of the composition, preferably between 0.2% and 3.0% by weight, preferentially between 0.4% and 1.0% by weight, better still 0.5% by weight.

### Aqueous Phase

The aqueous phase of a composition according to the invention, besides the water, can optionally comprise at least one water-soluble solvent.

In the present invention, the term “water-soluble solvent” denotes a compound which is liquid at ambient temperature and miscible with water (miscibility in water of greater than 50% by weight at 25° C. and atmospheric pressure).

The water-soluble solvents which can be used in the composition of the invention can in addition be volatile.

Mention may in particular be made, among the water-soluble solvents which can be used in the composition in accordance with the invention, of lower monoalcohols having from 1 to 5 carbon atoms, such as ethanol and isopropanol, C₃ and C₄ ketones, C₂-C₄ aldehydes, and also the polyols as defined in the present invention.

The aqueous phase (water and optionally the water-miscible solvent) can be present in the composition in a content ranging from 30% to 98% by weight, better still from 35% to 97% by weight and preferably from 40% to 95% by weight, with respect to the total weight of said composition.

According to an alternative embodiment, the aqueous phase of a composition according to the invention comprises at least water.

According to another alternative embodiment, the aqueous phase of a composition according to the invention can comprise water and at least one polyol as defined in the present invention, in particular C₂-C₃₂ polyol.

According to another alternative embodiment, the aqueous phase of a composition according to the invention can comprise water and at least one lower monoalcohol having from 1 to 5 carbon atoms.

According to another alternative embodiment, the aqueous phase of a composition according to the invention can comprise water, at least one lower monoalcohol having from 1 to 5 carbon atoms and at least one polyol as defined in the present invention, in particular C₂-C₃₂ polyol.

Depending on the presentation form or, when the composition is in the form of an emulsion, depending on the sense of the emulsion, the aqueous phase can be composed of an unmodified clay suitable for the invention in gel form, alone or in combination with other gelling agents.

As set out above, an unmodified clay suitable for the invention forms an aqueous gel in the presence of water. This aqueous gel can then constitute all or part of the aqueous phase. As such, it acts as rheological agent, as stabilizer of the emulsion. Thus, the stability of the final composition is improved. This property also applies when the unmodified clay gel suitable for the invention is used in combination with other aqueous gelling agents.

According to a specific embodiment, an unmodified clay suitable for the invention in the aqueous gel form constitutes the aqueous phase of a composition according to the invention, that is to say that the aqueous phase of the composition is exclusively constituted of this gel.

Oily Phase

When the composition used according to the invention includes an oily phase, the latter preferably contains at least one oil, in particular a cosmetic oil. It can additionally contain other fatty substances, such as crystallizable fatty substances as defined in the present invention or distinct from these.

Mention may be made, as oils which can be used in the composition of the invention, for example, of:
hydrocarbon oils of animal origin, such as perhydrosqualene;
hydrocarbon oils of vegetable origin, such as liquid triglycerides of fatty acids comprising from 4 to 10 carbon atoms, such as triglycerides of heptanoic or octanoic acids, or also, for example, sunflower oil, corn oil, soybean oil, pumpkin oil, grapeseed oil, sesame oil, hazelnut oil, apricot oil, macadamia oil, arara oil, castor oil, avocado oil, caprylic/capric acid triglycerides, such as those sold by Stéarinerie Dubois or those sold under the names Miglyol 810, 812 and 818 by Dynamit Nobel, jojoba oil or shea butter oil;
hydroxylated esters, such as isostearyl lactate, octyl hydroxystearate, octyldodecyl hydroxystearate, diisostearyl malate, triisocetyl citrate or heptanoates, octanoates or decanoates of fatty alcohols;
polyol esters, such as propylene glycol dioctanoate, neopentyl glycol diheptanoate and diethylene glycol diisononanoate;
pentaerythritol esters, such as pentaerythrityl tetraisostearate;
linear or branched hydrocarbons of vegetable, mineral or synthetic origin, such as volatile or nonvolatile liquid paraffins, and their derivatives, hydrocarbon oils having a branched chain comprising from 10 to 20 carbon atoms, such as isohexadecane, isododecane, isoparaffins and their mixtures, petrolatum, polydecenes or hydrogenated polyisobutene, such as Parleam® oil;
partially hydrocarbon-based and/or silicone-based fluorinated oils, such as those described in the document JP 2295912;
silicone oils, such as volatile or nonvolatile polymethylsiloxanes (PDMSs) having a linear or cyclic silicone chain, which are liquid or pasty at ambient temperature, in particular cyclopolydimethylsiloxanes (cyclomethicones), such as cyclohexasiloxane; polydimethylsiloxanes comprising alkyl, alkoxy or phenyl groups, which are pendent or at the end of the silicone chain, which groups have from 2 to 24 carbon atoms; phenylated silicones, such as phenyl trimethicones, phenyl dimethicones, phenyl(trimethylsiloxy)diphenylsiloxanes, diphenyl dimethicones, diphenyl(methyldiphenyl)trisiloxanes, (2-phenylethyl)trimethylsiloxy silicates and polymethylphenylsiloxanes; and
their mixtures.

In the list of the abovementioned oils, the term “hydrocarbon oil” is understood to mean any oil predominantly comprising carbon and hydrogen atoms, and optionally ester, ether, fluoro, carboxylic acid and/or alcohol groups.

The other fatty substances which can be present in the oily phase are, for example, silicone resins, such as trifluoromethyl(C₁-C₄)alkyl dimethicone and trifluoropropyl dimethicone, and silicone elastomers, such as the products sold under the KSG names by Shin-Etsu, under the Trefil, BY29 or names by Dow Corning or under the Gransil names by Grant Industries.

These fatty substances can be chosen in a way varied by a person skilled in the art in order to prepare a composition having the desired properties, for example of consistency or of texture.

According to one alternative form, the amount of the oily phase can range, for example, from 1% to 70% by weight and, for example, from 5% to 50% by weight or, for example, from 5% to 20% by weight, with respect to the total weight of the composition.

According to one alternative form, the amount of oily phase can range, for example, from 5% to 10% by weight, with respect to the total weight of the composition.

Pasty Compound

Within the meaning of the present invention, the term “pasty” is understood to denote a lipophilic fatty compound having a reversible solid/liquid change in state and comprising, at the temperature of 23° C., a liquid fraction and a solid fraction.

The pasty compound is advantageously chosen from:
lanolin and its derivatives,
polymeric or nonpolymeric fluorinated compounds,
polymeric or nonpolymeric silicone compounds,
vinyl polymers, in particular:
olefin homopolymers,
olefin copolymers,
hydrogenated diene homopolymers and copolymers,
linear or branched homo- or copolymer oligomers of alkyl (meth)acrylates preferably having a C₈-C₃₀ alkyl group,
homo- and copolymer oligomers of vinyl esters having C₈-C₃₀ alkyl groups,
homo- and copolymer oligomers of vinyl ethers having C₈-C₃₀ alkyl groups,
liposoluble polyethers resulting from the polyetherification between one or more C₂-C₁₀₀ and preferably C₂-050 diols,
esters,
polyvinyl laurate, and
their mixtures.

By way of indication, a composition according to the invention can comprise from 0% to 10% by weight, better still from 1% to 5% by weight, of pasty compound(s), with respect to the total weight of the composition.

Other Additives

The cosmetic composition can additionally comprise at least one additive chosen from the adjuvants normal in the cosmetics field, such as hydrophilic or lipophilic gelling agents, water-soluble or fat-soluble active agents, for example antiaging active agents, film-forming polymers, preservatives, sequestering agent, antioxidants, solvents, fragrances, odor absorbers, pH adjusters (acids or bases), surface-active agents, colorants (intended to color the composition according to the invention) and their mixtures.

According to a specific embodiment, the composition can comprise at least one hydrotropic molecule, such as, for example, nicotinamide (vitamin B3), caffeine, sodium PCA, sodium salicylate, urea, (hydroxyethyl)urea and any one of their mixtures.

According to a specific embodiment of the invention, the composition can comprise at least one active agent. In particular, the active agent can be present in a composition according to the invention in a content of between 0.001% and 10% by weight, preferably of between 0.01% and 5% by weight, with respect to the total weight of the composition.

According to one embodiment, the composition according to the invention can additionally comprise at least one preservative.

According to one embodiment, the composition additionally comprises a fragrance. According to one alternative form, the amount of fragrances can range, for example, from 0.001% to 10% and preferably from 0.01% to 5%, by weight, with respect to the total weight of the composition.

A person skilled in the art will take care to choose the optional additional adjuvants and/or their amounts so that the advantageous properties of the composition are not, or not substantially, detrimentally affected by the envisaged addition.

The compositions, in particular cosmetic compositions, according to the invention comprise a physiologically acceptable medium.

Within the meaning of the present invention, the term “physiologically acceptable medium” is understood to denote a medium suitable for the administration of a composition by the topical route.

A physiologically acceptable medium is generally without odor, or unpleasant appearance, and is entirely compatible with the topical administration route. In the present case, where the composition is intended to be administered by the topical route, that is to say by application at the surface of the keratin material under consideration, such a medium is in particular regarded as physiologically acceptable when it does not cause tingling, tautness or redness unacceptable to the user.

In particular, the composition is suited to topical application, that is to say to application at the surface of the skin, the scalp and/or the mucus membrane under consideration.

Thus, the physiologically acceptable medium is preferentially a cosmetically or dermatologically acceptable medium, that is to say a medium without unpleasant odor, color or appearance, and which does not cause tingling, tautness or redness unacceptable to the user.

The composition can then comprise all the constituents normally employed in the application envisaged.

Of course, a person skilled in the art will take care to choose this or these optional additional compounds, and/or their amounts, so that the advantageous properties of the compounds according to the invention are not, or not substantially, detrimentally affected by the envisaged addition.

Composition

The compositions according to the invention can be prepared according to the techniques well known to a person skilled in the art.

The composition according to the invention can be provided in any presentation form conventionally used according to the applications envisaged.

For example, when the composition according to the invention is cosmetic or dermatological, it can be provided in any presentation form conventionally employed for topical applications and in particular in the form of a dispersion of aqueous lotion or gel type, of an emulsion of liquid to semisolid consistency, obtained by dispersion of a fatty phase in an aqueous phase (O/W) or vice versa (W/O), or of a liquid to semisolid suspension of emulsified cream or gel type.

Preferably, the composition is provided in the form of an oil-in-water (direct emulsion (O/W)) or water-in-oil (inverse emulsion (W/O)) emulsion, preferably an oil-in-water emulsion, a gel or an emulsified gel.

According to a specific embodiment, a composition according to the invention is of gel type.

According to one embodiment, a composition of the invention can advantageously be provided in the form of a composition for caring for the skin of the body or of the face, in particular of the face, or for caring for the nails.

Thus, the compositions according to the invention can be in the form of products for caring for the skin or semimucus membranes, such as a protective or cosmetic care composition for the face, for the lips, for the hands, for the feet, for the anatomical folds or for the body (for example, day creams, night cream, day serum, night serum, antisun composition, protective or care body milk, aftersun milk, lotion, gel or foam for the care of the skin or of the scalp, serum, mask or aftershave composition).

According to another embodiment, a composition of the invention can advantageously be provided in the form of a composition for the hygiene of the skin of the body, in particular of the armpits or of the feet.

The compositions according to the invention can furthermore be packaged in pressurized form in an aerosol device or in a pump-action spray; packaged in a device equipped with an openwork wall, in particular a grating; packaged in a device equipped with a ball applicator (roll-on); or packaged in the form of sticks. In this regard, they contain the ingredients generally used in products of this type and which are well known to a person skilled in the art.

The compositions packaged as an aerosol in accordance with the invention generally contain conventional propellants, such as, for example, hydrofluorinated compounds, dichlorodifluoromethane, difluoroethane, dimethyl ether, isobutane, n-butane, propane or trichlorofluoromethane. They are preferably present in amounts ranging from 15% to 50% by weight, with respect to the total weight of the composition.

Such compositions are in particular prepared according to the general knowledge of a person skilled in the art.

A composition according to the invention preferably exhibits a viscosity of between 0.1 Pa·s and 2000 Pa·s, better still of between 1 Pa·s and 1000 Pa·s, even better still of between 10 Pa·s and 500 Pa·s.

The viscosity of the composition is measured at ambient temperature (25° C.) using a Haake MARS (Modular Advanced Rheometer System) rheometer equipped with a rotor to which the geometries are attached. In the context of the present invention, the cone/plate or plate/plate geometry has been used.

Preferably, a cream or a gel, in particular an aqueous gel, is concerned.

Advantageously, the pH of the composition is between 5 and 12 and preferably between 10 and 12.

According to a particular embodiment, the present invention is targeted at a composition, preferably a cosmetic composition, comprising at least:
an unmodified clay chosen from hectorite, saponite, stevensite and their mixtures,
a magnesium salt chosen from magnesium oxide, magnesium hydroxide and their mixtures,
an antisyneresis agent chosen from propylene glycol, 1,3-propanediol, and their mixtures, and
water.

According to another particular embodiment, the present invention is targeted at a composition, preferably a cosmetic composition, comprising at least:
an unmodified clay which is hectorite, and in particular natural hectorite,
a magnesium salt chosen from magnesium oxide, magnesium hydroxide and their mixtures,
an antisyneresis agent chosen from propylene glycol, 1,3-propanediol, and their mixtures, and
water.

According to another particular embodiment, the present invention is targeted at a composition, preferably a cosmetic composition, comprising at least:
an unmodified clay which is hectorite, and in particular natural hectorite,
a magnesium salt chosen from magnesium oxide and magnesium hydroxide,
an antisyneresis agent chosen from propylene glycol, 1,3-propanediol, and their mixtures, and
water.

According to another particular embodiment, the present invention is targeted at a composition, preferably a cosmetic composition, comprising at least:
an unmodified clay which is hectorite, and in particular natural hectorite,
a magnesium salt chosen from magnesium oxide and magnesium hydroxide,
an antisyneresis agent which comprises propylene glycol, and
water.

According to another particular embodiment, the present invention is targeted at a composition, preferably a cosmetic composition, comprising at least:
an unmodified clay which is hectorite, and in particular natural hectorite,
a magnesium salt which is magnesium oxide,
an antisyneresis agent which is propylene glycol and
water.

According to another particular embodiment, the present invention is targeted at a composition, preferably a cosmetic composition, comprising at least:
an unmodified clay which is hectorite, and in particular natural hectorite,
a magnesium salt which is magnesium hydroxide,
an antisyneresis agent which is propylene glycol, and
water.

According to another particular embodiment, the present invention is targeted at a composition, preferably a cosmetic composition, comprising at least:
an unmodified clay chosen from hectorite, saponite, stevensite and their mixtures,
a magnesium salt chosen from magnesium oxide, magnesium hydroxide and their mixtures,
an antisyneresis agent chosen from propylene glycol, 1,3-propanediol, and their mixtures, and
water,
wherein said antisyneresis agent is present in an amount ranging from 3% to 50% by weight, with respect to the total weight of the composition.

According to another particular embodiment, the present invention is targeted at a composition, preferably a cosmetic composition, comprising at least:
an unmodified clay which is hectorite, and in particular natural hectorite,
a magnesium salt chosen from magnesium oxide, magnesium hydroxide and their mixtures,
an antisyneresis agent chosen from propylene glycol, 1,3-propanediol, and their mixtures, and
water,
wherein said antisyneresis agent is present in an amount ranging from 3% to 50% by weight, with respect to the total weight of the composition.

According to another particular embodiment, the present invention is targeted at a composition, preferably a cosmetic composition, comprising at least:
an unmodified clay which is hectorite, and in particular natural hectorite,
a magnesium salt chosen from magnesium oxide and magnesium hydroxide,
an antisyneresis agent chosen from propylene glycol, 1,3-propanediol, and their mixtures, and
water,
wherein said antisyneresis agent is present in an amount ranging from 3% to 50% by weight, with respect to the total weight of the composition.

According to another particular embodiment, the present invention is targeted at a composition, preferably a cosmetic composition, comprising at least:
an unmodified clay which is hectorite, and in particular natural hectorite,
a magnesium salt chosen from magnesium oxide and magnesium hydroxide,
an antisyneresis agent which comprises propylene glycol, and
water,
wherein said antisyneresis agent is present in an amount ranging from 3% to 50% by weight, with respect to the total weight of the composition.

According to another particular embodiment, the present invention is targeted at a composition, preferably a cosmetic composition, comprising at least:
an unmodified clay which is hectorite, and in particular natural hectorite,
a magnesium salt which is magnesium oxide,
an antisyneresis agent which is propylene glycol and
water,
wherein said antisyneresis agent is present in an amount ranging from 3% to 50% by weight, with respect to the total weight of the composition.

According to another particular embodiment, the present invention is targeted at a composition, preferably a cosmetic composition, comprising at least:
an unmodified clay which is hectorite, and in particular natural hectorite,
a magnesium salt which is magnesium hydroxide,
an antisyneresis agent which is propylene glycol, and
water;
wherein said antisyneresis agent is present in an amount ranging from 3% to 50% by weight, with respect to the total weight of the composition.

The expressions “between . . . and . . . ” and “ranging from . . . to . . . ” should be understood as meaning limits included, unless otherwise specified.

In the description and the examples, unless otherwise indicated, the percentages are percentages by weight. The percentages are thus expressed by weight, with respect to the total weight of the composition. The temperature is expressed in degrees Celsius, unless otherwise indicated, and the pressure is atmospheric pressure, unless otherwise indicated.

The invention is illustrated in greater detail by the non-limiting examples presented below.

### EXAMPLE

### Example 1: Compositions 1 to 3 and 3a

Three compositions 1 to 3 and 3a of cream type based on unmodified hectorite were prepared from the compounds and contents given in detail in table 1 below. The contents are expressed as percentages by weight, with respect to the total weight of the composition.

More specifically, products for greasy skin are concerned.

TABLE 1

| | Formula 1 | Formula 2 | Formula 3 | Formula 3a |
|---|---|---|---|---|
| | according to | according to | according to | according to |
| Compounds | the invention | the invention | the invention | the invention |
| Hectorite (INCI name: | 3 | 5 | 4 | 5 |
| hectorite) Bentone EW | | | | |
| from Elementis Specialties | | | | |
| Magnesium oxide MgO (INCI name: | 0.9 | 0.9 | 0.9 | 0.9 |
| Magnesium Oxide) | | | | |
| Magnesium Oxide Extra Light Low | | | | |
| Nickel from Dr. Paul Lohmann | | | | |
| Cetearyl alcohol (INCI name: | 3 | — | — | — |
| Cetearyl Alcohol) | | | | |
| Lanette O OR from BASF | | | | |
| Cetyl esters (INCI name: | 0.5 | — | — | — |
| Cetyl Esters (and) Cetyl Esters) | | | | |
| SP Crodamol MS MBAL-PA-(SG) | | | | |
| from Croda | | | | |
| Stearic acid (INCI name: | 0.2 | — | — | — |
| Stearic Acid) | | | | |
| Stearin TP 1200 Pellets (DUB 50 P) | | | | |
| from Stéarinerie Dubois | | | | |
| Propylene glycol (INCI name: | — | — | 5 | 5 |
| Propylene Glycol) | | | | |
| 1,2-Propylene Glycol Care from BASF | | | | |
| Cellulose gum (INCI name: | 0.1 | — | — | — |
| Cellulose Gum) | | | | |
| Aquasorb A 500 from Ashland | | | | |
| Sclerotium gum (INCI name: | — | — | 0.2 | 0.2 |
| Sclerotium gum) | | | | |
| Actigum CS 11 QD from Cargill | | | | |
| Gum arabic (INCI name: Acacia | — | 10 | — | — |
| Senegal Gum) | | | | |
| Spray-dried Gum Acacia 396A | | | | |
| from Alland & Robert | | | | |
| Phenoxyethanol (INCI name: | 0.5 | 0.5 | 0.5 | 0.5 |
| Phenoxyethanol) | | | | |
| Phenoxetol from Clariant | | | | |
| Methylparaben (INCI name: | 0.1 | 0.1 | 0.1 | 0.1 |
| Methylparaben) | | | | |
| Nipagin M from Clariant | | | | |
| Water | q.s. for 100 | q.s. for 100 | q.s. for 100 | q.s. for 100 |
| pH | 10.8 | 10.8 | 10.8 | 10.8 |

The compositions are prepared according to the protocol indicated below:
the unmodified hectorite is introduced into the water with vigorous stirring at a temperature of 60° C.,
the following ingredients (methylparaben and the polymer) are introduced into the preceding mixture with stirring,
a supplement is prepared with the molten fatty substances,
once the aqueous hectorite gel is formed, the molten fatty substances are added with stirring,
the temperature of the preparation is lowered down to ambient temperature, still with stirring,
phenoxyethanol is then added,
the magnesium salt MgO is added at the end, with stirring.

The compositions 1 to 3 and 3a in accordance with the invention were stable. No liquid phase ejected from the system was visible to the naked eye, which confirms that no syneresis occurred.

Mattness Performance

In order to demonstrate the persistence of the mattness over time, the mattness of the formulae 2 and 3a and of a product referent test was evaluated in vivo, at t=0 minutes and two hours after application. In order to do this, the composition is applied to small surface areas (4*3.5 cm) located on the forehand and the cheeks of the face of 28 users (N=28) having either greasy skin or mixed skin with a greasy tendency, and measurements of the mattness are carried out at t=0 minutes and at t=10 minutes. The users subsequently stay in a steam room for 2 hours and further measurements are taken.

The product referent test is named normaderm gel cream base (2% aerogel) which comprises water, glycerin, propylene glycol, AMMONIUM POLYACRYLOYLDIMETHYL TAURATE, DIMETHICONE, PEG-12 DIMETHICONE, BIOSACCHARIDE GUM-1, SILICA SILYLATE, CAPRYLYL GLYCOL, MENTHOL, ALCOHOL DENAT, and fragrance.

Results

Immediate Effectiveness of Compositions 2 and 3a According to the Invention Compared to the Reference

Gc (contrast gloss) is the difference in apparent luminosity in the specular and far from the specular. It is defined for medium glossy surfaces in which the image of the illumination source is not visible (such as skin). The effectiveness was compared after 10 minutes. The following formula was used (the two compositions 2 and 3a are named “product”):

(Tᵢₘₘ−T₀)_{product} versus(Tᵢₘₘ−T₀)_{reference}

The results are gathered in the following table 1a:

TABLE 1a

| | | | Reference | |
|---|---|---|---|---|
| | | | Normaderm | |
| | Composition 2 | Composition 3a | (2% aerogel) | P-value |
| Parameter | (Mean ± 95% IC) | (Mean ± 95% IC) | (Mean ± 95% IC) | N = 28 |
| Gc | −13.12 ± 1.21 | −14.26 ± 1.21 | −9.5 ± 1.21 | S (p < 0.001) |
| Contrast gloss | | | | |

IC: Confidence Interval; S: statistical significance; p-value: probability value.

It comes out from these results that the immediate effectiveness of compositions 2 and 3a according to the invention is significantly better than that of the reference.

More particularly, compositions 2 and 3a according to the invention significantly modify/decrease the shine of the skin as soon as it is applied by matifying it.

Mattness Resistance in Extreme Conditions (in a Steam Room for 2 Hours) of Compositions 2 and 3a According to the Invention Compared to the Reference and Bare Skin

Evolution of the compositions 2 and 3a, and reference (2% aerogel) was compared to that of bare skin. The following formula was used (the three compositions 2, 3a and reference are named “product”):

(T₂ₕ−T₀)_{product} versus(T₂ₕ−T₀)_{bare skin}
T₂ₕ is after sweating.

The results are gathered in the following table 1b:

TABLE 1b

| | | | Reference | | |
|---|---|---|---|---|---|
| | | | Normaderm | | |
| | Composition 2 | Composition 3a | (2% aerogel) | Bare skin | P-value |
| Parameter | (Mean ± 95% IC) | (Mean ± 95% IC) | (Mean ± 95% IC) | (Mean ± 95% IC) | N = 28 |
| Gc | −10.83 ± 1.58 | −12.31 ± 1.58 | −8.05 ± 1.58 | 9.13 ± 1.58 | S (p < 0.0099) |
| Contrast gloss | | | | | |

IC: Confidence Interval; S: statistical significance; p-value: probability value.

First, it comes out from these results that the reference Normaderm (2% aerogel) limits shine as its evolution is significantly different from that of bare skin.

Furthermore, after 2 hours in a steam room, compositions 2 and 3a in accordance with the invention limit significantly the shine in comparison with bare skin.

In addition, after 2 hours in a steam room, the ability of compositions 2 and 3a in accordance with the invention to limit shine is significantly better than that of the reference.

Further, compositions 2 and 3a in accordance with the invention allow the preservation of the initial level of shine after 2 hours in a steam room, even a mattifying effect and in spite of sweating.

In conclusion, the results show that the formulae 2 and 3a according to the invention provide a mattifying effect, in other words they significantly limit the shininess of the skin in comparison with bare skin. Furthermore, it makes possible greater persistence of the mattness after 2 hours, this being the case despite sweating.

In addition, whether in immediate effectiveness or after 2 hours in the steam room, the formulae 2 and 3a comprising different polymers do not behave differently.

Furthermore, advantageously, on application, the formulae 1 to 3 and 3a are fresh, light, nontacky and nonstringy. They spread easily without soaping.

### Example 2: Compositions 4 to 22

Nineteen compositions 4 to 22 of gel (balm or cream) type based on unmodified hectorite were prepared from the compounds and contents given in detail in tables 2 to 6 below. The contents are expressed as percentages by weight, with respect to the total weight of the composition.

More specifically, deodorant products are concerned.

TABLE 2

| | Formula 4 | Formula 5 | Formula 6 |
|---|---|---|---|
| | according to | according to | according to |
| | the invention | the invention | the invention |
| Compounds | Gel, balm | Gel, balm | Gel, balm |
| Hectorite (INCI name: | 5 | 5 | 5 |
| hectorite) Bentone EW | | | |
| from Elementis Specialties | | | |
| Magnesium oxide MgO | 2 | 2 | 2 |
| (INCI name: Magnesium Oxide) | | | |
| Magnesium Oxide Extra Light | | | |
| Low Nickel from Dr. Paul Lohmann | | | |
| Sclerotium gum (INCI name: | 0.1 | 0.1 | 0.1 |
| Sclerotium gum) | | | |
| Actigum CS 11 QD from Cargill | | | |
| Propylene glycol (INCI name: | 20 | — | 50 |
| Propylene Glycol) | | | |
| 1,2-Propylene Glycol Care | | | |
| from BASF | | | |
| Water | q.s. for 100 | q.s. for 100 | q.s. for 100 |
| pH | 12.3 | 12.1 | 12.4 |

TABLE 3

| | Formula 7 | FIG. 8 | Formula 9 | Formula 10 |
|---|---|---|---|---|
| | according to | according to | according to | according to |
| | the invention | the invention | the invention | the invention |
| Compounds | Gel, cream | Gel, cream | Gel, balm | Gel, balm |
| Hectorite (INCI name: | 1.25 | 1.25 | 2.5 | 4 |
| hectorite) Bentone EW | | | | |
| from Elementis Specialties | | | | |
| Magnesium oxide MgO | 4 | 4 | 4 | 4 |
| (INCI name: Magnesium Oxide) | | | | |
| Magnesium Oxide Extra Light Low | | | | |
| Nickel from Dr. Paul Lohmann | | | | |
| Propylene glycol (INCI | 35 | 20 | 30 | 30 |
| name: Propylene Glycol) | | | | |
| 1,2-Propylene Glycol | | | | |
| Care from BASF | | | | |
| Water | q.s. for 100 | q.s. for 100 | q.s. for 100 | q.s. for 100 |
| pH | 12 | 12 | 12 | 12 |

TABLE 4

| | Formula 11 | Formula 12 | Formula 13 | Formula 14 |
|---|---|---|---|---|
| | according to | according to | according to | according to |
| | the invention | the invention | the invention | the invention |
| Compounds | Gel, balm | Gel, balm | Gel, balm | Gel, balm |
| Hectorite (INCI name: hectorite) | 3 | 5 | 5 | 5 |
| Bentone EW from Elementis | | | | |
| Specialties | | | | |
| Magnesium oxide MgO (INCI | 2 | — | — | 1 |
| name: Magnesium Oxide) | | | | |
| Magnesium Oxide Extra Light | | | | |
| Low Nickel from Dr. Paul Lohmann | | | | |
| Magnesium hydroxide (INCI | — | 10 | 2 | — |
| name: Magnesium Hydroxide) | | | | |
| Magnesium Hydroxide from | | | | |
| Dr. Paul Lohmann | | | | |
| Cetyl esters (INCI name: Cetyl | 0.5 | — | — | — |
| Esters (and) Cetyl Esters) | | | | |
| SP Crodamol MS MBAL-PA-(SG) | | | | |
| from Croda | | | | |
| Cetearyl alcohol (INCI name: | 3 | — | — | — |
| Cetearyl Alcohol) | | | | |
| Lanette O OR from BASF | | | | |
| Stearic acid (INCI name: | 0.2 | — | — | — |
| Stearic Acid) | | | | |
| Stearin TP 1200 Pellets | | | | |
| (DUB 50 P) from | | | | |
| Stéarinerie Dubois | | | | |
| Propylene glycol (INCI name: | 20 | 20 | 20 | 20 |
| Propylene Glycol) | | | | |
| 1,2-Propylene Glycol Care from | | | | |
| BASF | | | | |
| Water | q.s. for 100 | | q.s. for 100 | q.s. for 100 |
| pH | 12 | 11 | 11 | 12 |

TABLE 5

| | Formula 15 | FIG. 16 | Formula 17 | Formula 18 |
|---|---|---|---|---|
| | according to | according to | according to | according to |
| | the invention | the invention | the invention | the invention |
| Compounds | Gel, balm | Gel, balm | Gel, balm | Gel, cream |
| Hectorite (INCI name: | 5 | 5 | 5 | 2 |
| hectorite) Bentone EW | | | | |
| from Elementis Specialties | | | | |
| Magnesium oxide MgO | — | — | — | 8 |
| (INCI name: Magnesium Oxide) | | | | |
| Magnesia 22 from Magnesia | | | | |
| Magnesium hydroxide | 3 | 5 | 7 | — |
| (INCI name: Magnesium Hydroxide) | | | | |
| Magnesium Hydroxide from | | | | |
| Dr. Paul Lohmann | | | | |
| Propylene glycol (INCI | 20 | 20 | 20 | 20 |
| name: Propylene Glycol) | | | | |
| 1,2-Propylene Glycol Care | | | | |
| from BASF | | | | |
| Water | q.s. for 100 | q.s. for 100 | q.s. for 100 | q.s. for 100 |
| pH | 10.5 | 10.5 | 10.5 | 12 |

TABLE 6

| | Formula 19 | FIG. 20 | Formula 21 | Formula 22 |
|---|---|---|---|---|
| | according to | according to | according to | according to |
| | the invention | the invention | the invention | the invention |
| Compounds | Gel, balm | Gel, balm | Gel, balm | Gel, balm |
| Hectorite (INCI name: | 5 | 5 | 5 | 5 |
| hectorite) Bentone EW | | | | |
| from Elementis Specialties | | | | |
| Magnesium oxide MgO | 2 | 4 | 6 | 8 |
| (INCI name: Magnesium Oxide) | | | | |
| Magnesia 22 from Magnesia | | | | |
| Propylene glycol (INCI | 20 | 20 | 20 | 20 |
| name: Propylene Glycol) | | | | |
| 1,2-Propylene Glycol Care | | | | |
| from BASF | | | | |
| Water | q.s. for 100 | q.s. for 100 | q.s. for 100 | q.s. for 100 |
| pH | 12 | 12 | 12 | 12 |

The compositions are prepared according to the protocol indicated below:
the unmodified hectorite is introduced into the water with vigorous stirring at a temperature of 60° C.,
the other ingredients are introduced into the preceding mixture with stirring,
once the aqueous hectorite gel is formed, the magnesium salt MgO or Mg(OH)₂ is added with stirring.

The compositions 4 to 22 in accordance with the invention were stable. No liquid phase ejected from the system was visible to the naked eye, which confirms that no syneresis occurred.

### Deodorant Performance

For this test, a comparative formula of a product already marketed, Roll On Cadum Emulsion, with 1% of ZnGlu (zinc gluconate) was used. This comparative formula comprises water, butylene glycol, ZINC GLUCONATE, CITRIC ACID, CAPRYLYL GLYCOL, ALLANTOIN, P-ANISIC ACID, PPG-15 STEARYL ETHER, DIMETHICONE, CETEARETH-33, CETEARYL ALCOHOL, PERLITE, and talc.

The deodorant effectiveness of several formulae illustrated in tables 5 and 6 was evaluated according to the following protocol:

The two armpits of each person (13 persons) were first washed (after wetting) with a dilute shower gel (broad area) while causing it to foam for 20 seconds. The armpits were copiously rinsed and then dried by lightly patting. The washing was carried out a second time for each of the armpits.

0.5 g of formula was then applied to the surface of just one of the two armpits via a device of roll-on type.

One armpit has thus not been treated and serves as reference for the evaluation.

After 24 hours, the evaluations are carried out by a direct “sniff test” of the intensity of the odor under the armpits after a single application of the test composition compared with the intensity of the odor under the untreated armpit.

The deodorant effectiveness was evaluated according to the following criterion:

The intensity of the odor on a continuous linear scale from 0 (“no odor”) to 100 (“extreme odor”) with a reference point at 50 (“perspiration odor”).

The results are summarized in table 7.

TABLE 7

| Formulae | Intensity of the odor |
|---|---|
| Formula 15 | 19 |
| Formula 16 | 20 |
| Formula 17 | 17 |
| Formula 18 | 11 |
| Formula 19 | 19 |
| Formula 20 | 18 |
| Formula 21 | 15 |
| Formula 22 | 15 |
| Roll-On Cadum Emulsion with 1% | 27 |
| of ZnGlu - comparative formula | |

It emerges from these results that the compositions according to the invention provide a superior deodorant effect to the comparative formula of Roll-On Cadum Emulsion with 1% of ZnGlu insofar as intensity of the odor is lower after 24 hours.

### Other Tests of Evaluation of the Intensity of the Perspiration Odor, “Sniff Test”

For these tests, the same comparative formula as that of the test above (product Roll-On Cadum Emulsion with 1% of ZnGlu) was used.

The deodorant effectiveness of the three formulae 4, 5 and 6 which are illustrated in table 2 above was evaluated according to the following protocol:

The 22 subjects (age 48-56 years) selected had a mean odor intensity under the armpits of greater than or equal to 5 (in the case in point, 6.1-6.4) over a scale ranging from 1 (no odor) to 10 (excessively strong odor) and a mean difference in intensity between the two armpits of less than or equal to 1 (in the case in point, 0.4-0.6).

The two armpits of each person were washed for a week before the start of the test with a neutral washing product. On the day of the test, the armpits (treated and untreated) were dried before application of the product. One of the deodorant formulae is then applied to an armpit. There were in all 4 applications.

The comparison is thus carried out between a treated armpit versus a treated or untreated armpit.

When an application advice of roll-on type is concerned, 0.4 g (±0.05 g) is applied per day per armpit.

When an application advice of aerosol type is concerned, 1.2 g (±0.05 g) is applied per day per armpit.

The direct evaluation of the sniffing (“sniff”) of the intensity of the perspiration odor is subsequently carried out respectively 8 h and then 24 h after application of the deodorant product.

The deodorant effectiveness was evaluated according to the following criterion:

The intensity of the odor on a continuous linear scale from 0 (“no odor”) to 10 (“excessively strong odor”).

The percentage variations are calculated over the mean according to the following equation:

$$Δ \% = \frac{\begin{matrix}treated armpit A - \\ treated armpit B or untreated armpit * 100\end{matrix}}{treated armpit B or untreated armpit} \qquad [ Math 1 ]$$

The variability makes it possible to determine the dynamics of response of the volunteers.

This is calculated according to the following mathematical formula:

$$Variability = \frac{SD * 100}{mean} \qquad [ Math 2 ]$$

in which SD means standard deviation

The comparative results, respectively for the formulae 4, 5 and 6, are summarized in tables 8, 9 and 10.

TABLE 8

| | Intensity of the odor 8 | | Intensity of the odor 24 | |
|---|---|---|---|---|
| | hours after 4 applications | | hours after 4 applications | |
| | | Comparative | | Comparative |
| | | formula | | formula |
| | | Roll-On Cadum | | Roll-On Cadum |
| | | Emulsion with | | Emulsion with |
| | Formula 4 | 1% of ZnGlu | Formula 4 | 1% of ZnGlu |
| Mean | 3.3 | 5.3 | 4.2 | 5.9 |
| Standard deviation | 1.2 | 1.6 | 1.3 | 1.3 |
| Variability | 38% | 29% | 32% | 22% |

| | | |
|---|---|---|
| Δ (formula 4 - | −2.0 (S) | −1.7 (S) |
| comparative formula) | | |

(S): significative difference

8 hours after application, the intensity of the perspiration odor is evaluated as “not very intense” for the armpit treated with the formula 4 according to the invention and as “perceptible intense” for the armpit treated with the comparative formula.
24 hours after application, the intensity of the perspiration odor is evaluated as “moderately intense” for the armpit treated with the formula 4 according to the invention and as “rather intense” for the armpit treated with the comparative formula.

It thus emerges from these results that there exists a significant difference in the intensity of the perspiration odor between the two formulae, and that this difference is in favor of the formula according to the invention, whether 8 hours or 24 hours after application of the formulae (after 4 applications).

TABLE 9

| | Intensity of the odor 8 | | Intensity of the odor 24 | |
|---|---|---|---|---|
| | hours after 4 applications | | hours after 4 applications | |
| | | Comparative | | Comparative |
| | | formula | | formula |
| | | Roll-On Cadum | | Roll-On Cadum |
| | | Emulsion with | | Emulsion with |
| | Formula 5 | 1% of ZnGlu | Formula 5 | 1% of ZnGlu |
| Mean | 4.1 | 5.9 | 5.0 | 6.0 |
| Standard deviation | 1.3 | 1.7 | 1.5 | 1.5 |
| Variability | 31% | 29% | 30% | 26% |

| | | |
|---|---|---|
| Δ (formula 5 - | −1.8 (S) | −1.0 (S) |
| comparative formula) | | |

(S): significative difference

8 hours after application, the intensity of the perspiration odor is evaluated as “moderately intense” for the armpit treated with the formula 5 according to the invention and as “rather intense” for the armpit treated with the comparative formula.
24 hours after application, the intensity of the perspiration odor is evaluated as “perceptible intense” for the armpit treated with the formula 5 according to the invention and as “rather intense” for the armpit treated with the comparative formula.

It thus emerges from these results that there exists a significant difference in the intensity of the perspiration odor between the two formulae, and that this difference is in favor of the formula according to the invention, whether 8 hours or 24 hours after application of the formulae (after 4 applications).

TABLE 10

| | Intensity of the odor 8 | | Intensity of the odor 24 | |
|---|---|---|---|---|
| | hours after 4 applications | | hours after 4 applications | |
| | | Comparative | | Comparative |
| | | formula | | formula |
| | | Roll-On | | Roll-On |
| | | Cadum | | Cadum |
| | Formula | Emulsion with | Formula | Emulsion with |
| | 6 | 1% of ZnGlu | 6 | 1% of ZnGlu |
| Mean | 3.4 | 4.8 | 3.9 | 4.9 |
| Standard | 1.2 | 1.4 | 1.5 | 1.6 |
| deviation | | | | |
| Variability | 36% | 29% | 38% | 32% |
| Δ (formula | −1.4 (S) | | −1.0 (S) | |
| 6-comparative | | | | |
| formula) | | | | |

(S): significative difference

(S): significative difference
8 hours after application, the intensity of the perspiration odor is evaluated as “not very intense to moderately intense” for the armpit treated with the formula 6 according to the invention and as “perceptible intense” for the armpit treated with the comparative formula.
24 hours after application, the intensity of the perspiration odor is evaluated as “moderately intense” for the armpit treated with the formula 6 according to the invention and as “perceptible intense” for the armpit treated with the comparative formula.

It thus emerges from these results that there exists a significant difference in the intensity of the perspiration odor between the two formulae, and that this difference is in favor of the formula according to the invention, whether 8 hours or 24 hours after application of the formulae (after 4 applications).

Furthermore, on application, the formulae 4 to 22 are fresh, light, nontacky and nonstringy.

They spread easily without soaping.

### Example 3: Compositions 23 to 26

Four compositions 23 and 24 according to the invention and 25 and 26 out of invention of gel (balm) type based on unmodified hectorite were prepared from the compounds and contents given in detail in table 11 below. The contents are expressed as percentages by weight, with respect to the total weight of the composition.

TABLE 11

| | Formula 23 | FIG. 24 | Formula 25 | Formula 26 |
|---|---|---|---|---|
| | according to | according to | out of | out of |
| | the invention | the invention | invention | invention |
| Compounds | Gel, balm | Gel, balm | Gel, balm | Gel, balm |
| Hectorite (INCI name: | 3 | 3 | 3 | 3 |
| hectorite) Bentone EW | | | | |
| from Elementis Specialties | | | | |
| Magnesium oxide MgO | 1 | 1 | 1 | 1 |
| (INCI name: Magnesium Oxide) | | | | |
| Magnesium Oxide Extra Light Low | | | | |
| Nickel from Dr. Paul Lohmann | | | | |
| Propylene glycol (INCI | 20 | 50 | — | — |
| name: Propylene Glycol) | | | | |
| 1,2-Propylene Glycol Care | | | | |
| from BASF | | | | |
| Butylene glycol (INCI | — | — | 20 | 50 |
| name: Butylene Glycol) | | | | |
| 1,3-Butylene Glycol from Alzo | | | | |
| Water | q.s. for 100 | q.s. for 100 | q.s. for 100 | q.s. for 100 |
| pH | 12 | 12 | 12 | 12 |

The compositions are prepared according to the protocol indicated below:
the unmodified hectorite is introduced into the water with vigorous stirring at a temperature of 60° C.,
once the aqueous hectorite gel is formed, the other ingredients (propylene glycol or butylene glycol) are introduced into the preceding mixture with stirring,
the magnesium salts are added with stirring.

The compositions 23 to 24 in accordance with the invention were stable. No liquid phase ejected from the system was visible to the naked eye, which confirms that no syneresis occurred.

The compositions 23 to 24 in accordance with the invention have clean appearance, and are odorless and no sticky compared to compositions 25 and 26 out of invention which have strong odor and are sticky.

In addition, on application, the formulae 23 and 24 are fresh, light, nontacky and nonstringy.

They spread easily without soaping.

1. A composition, comprising at least:
an unmodified clay chosen from trioctahedral smectites,
a magnesium salt chosen from magnesium oxide, magnesium hydroxide and their mixtures,
an antisyneresis agent chosen from propylene glycol, 1,3-propanediol, natural polymers and their mixtures, and
water.

2. The composition as claimed in claim 1, in which said unmodified clay comprises at least 30% by weight, of SiO₂, with respect to the total weight of the unmodified clay, and at least 10% by weight of MgO, with respect to the total weight of the unmodified clay.

3. The composition as claimed in claim 1, in which said unmodified clay has an SiO₂/MgO ratio by weight ranging from 1 to 3.

4. The composition as claimed in claim 1, in which said unmodified clay has the general molecular formula:
$${{[ {M}_{8 - x}^{4 +} {M}_{x}^{3 +} ]}^{tetra} [ {M}_{6 - y}^{2 +} {M}_{y}^{+} ]}^{octa} {{O}_{20} ( OH )}_{4} {X}_{\frac{x + y}{k}}^{k +} \cdot n {H}_{2} O$$
in which:
M⁴⁺ represents a cation,
M³⁺ represents a cation,
M²⁺ represents a cation,
M⁺ represents a cation,
X represents an interfoliar cation,
x represents the degree of tetrahedral substitution,
y represents the degree of octahedral substitution,
k represents the valency of the interfoliar cation X, and
n represents an integer.

5. The composition as claimed in claim 1, in which said unmodified clay is chosen from hectorite, saponite, stevensite and their mixtures.

6. The composition as claimed in claim 1, in which said unmodified clay is natural.

7. The composition as claimed in claim 1, in which said unmodified clay is present in a content of less than or equal to 10% by weight with respect to the total weight of the composition.

8. The composition as claimed in claim 1, in which said magnesium salt is magnesium oxide.

9. The composition as claimed in claim 1, in which said magnesium salt(s) is (are) present in an amount of between and 10.0% by weight with respect to the total weight of the composition.

10. The composition as claimed in claim 1, in which the unmodified clay/magnesium salt(s) ratio by weight is between 10/1 and 1/10.

11. The composition as claimed in claim 1, in which said at least antisyneresis agent is chosen from propylene glycol, 1,3-propanediol, and their mixtures.

12. The composition as claimed in claim 1, in which said natural polymer is chosen from:
modified or native polysaccharides,
polyamino acids, and
their mixtures.

13. The composition as claimed in claim 1, comprising further at least one organic or nonorganic filler.

14. The composition as claimed in claim 1, comprising further at least one crystallizable fatty substance.

15. The composition as claimed in claim 1, in which, when the antisyneresis agent is propylene glycol, 1,3-propanediol or their mixtures, it is present in an amount ranging from 1% to 60% by weight with respect to the total weight of the composition;
or
when the antisyneresis agent is a natural polymer, it is present in an amount ranging from 0.05% to 10.00% by weight with respect to the total weight of the composition.

16. The composition as claimed in claim 1, in which the water is present in an amount ranging from 30% by weight to 98% by weight with respect to the total weight of the composition.

17. The composition as claimed in claim 1, which is of gel type.

18. A cosmetic method comprising at least one stage which consists in applying, to the skin and/or head hair a composition as defined according to claim 1.
